(19) United States
(12) Patent Application Publication
(10) Pub. No.: US 2004/0198704 A1
(43) Pub. Date: Oct. 7, 2004

(54) Compositions and method for treating microbial and parasitic infections in cattle and other animals

(75) Inventors: Dale E. Shuster, South Orange, NJ (US); David G. Sawutz, Maplewood, NJ (US); Kanwal J. Varma, Warren, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/816,663

(22) Filed: Apr. 2, 2004

Related U.S. Application Data
(60) Provisional application No. 60/460,126, filed on Apr. 3, 2003.

(51) Int. Cl. A61K 31/655; A61K 31/277; A61K 31/16; A61K 31/18

(52) U.S. Cl. 514/151; 514/522; 514/603; 514/630

(57) ABSTRACT

Novel formulations combining an antiparasitic compounds, such as an avermectin, with a fluorinated chloramphenicol or thiamphenicol derivative antibiotic such as Florfenicol are disclosed. Methods for using such formulations in the treatment and prevention of infectious diseases of bovines and swine, including bovine respiratory disease and parasitic infections are also disclosed.

### CROSS-REFERENCE TO RELATED APPLICATION

This application claims benefit of priority to U.S. Provisional Patent Application Serial No. 60/460,126 filed Apr. 3, 2003, the entirety of which is hereby incorporated by reference.

### FIELD OF THE INVENTION

The invention relates to compositions and methods for the treatment of bacterial infections, parasitic infections and parasitic infestations in animals. More particularly, the invention relates to a composition containing both an antibiotic and parasiticide for use in the treatment of bacterial infections, parasitic infections and parasitic infestations in animals such as cattle, sheep and swine.

### BACKGROUND OF THE INVENTION

All references cited herein are hereby incorporated in their entirety by reference.

Feedlots are in general use with beef cattle in the United States, Canada and other areas of the world. When cattle arrive at a feedlot, they are usually administered parasiticides to eradicate the internal and external parasites, which they acquired from pastures contaminated with helminth larvae, where the calves grazed prior to their shipment to the feedlot. Elimination of external and internal parasites at this time breaks the cycle of parasitism and prevents their spread to the other animals in the feedlot as calves and other animals are less likely to acquire parasites in the feedlot because they eat feeds that are free of parasites from bunks or mangers.

Without the use of parasiticides, internal and external parasites have been one of the most economically-important constraints in raising livestock. The damage worms cause ranges from decreased productivity of the animals to their death.

First line therapy for the treatment of parasitic worms is often carried out via the administration of an antiparasitic compound. One class of these compounds is avermectins. The avermectin family of compounds is a series of very potent antiparasitic agents known to be useful against a broad spectrum of endoparasites and ectoparasites in mammals.

In addition to the risks of parasitic infestation and widespread infection of cattle or other animals in the feedlot, the commingling of calves and other livestock from different sources causes the calves and other animals to be exposed to pathogens for which immunity has not developed. The stresses of shipping and change in diet reduces the calves' and other animals' immune defenses. Additionally the poor weather of autumn, when calves and other livestock are usually moved from pastures to feedlots, further increases the risk of illness. Together, these circumstances result in a high incidence of respiratory disease in the cattle or other animals when they first arrive at the feedlot and soon thereafter. It has become common to administer antimicrobial drugs to calves and other feedlot animals at the time of arrival into a feedlot, in order to reduce the incidence and severity of respiratory illness in the feedlot cattle and other stock.

Without the use of antimicrobial agents, bovine respiratory disease (BRD) has been one of the leading causes of economic loss to the cattle industry throughout the world. Excessive mortality, reduced weight gains, and the cost of treatment and prevention have placed a heavy burden on the industry.

Bovine respiratory disease (BRD) occurs in both dairy and beef cattle and is one of the leading causes of economic loss to the cattle industry throughout the world. These economic losses are due to excessive mortality, reduced weight gains as well as treatment and prevention costs. BRD is often referred to as the “bovine respiratory diseases complex” due to the multifactorial etiology.

The cost of death losses due to respiratory diseases vary around the world. Death losses in the U.S. are estimated to approach $1 billion annually. Losses in various European countries range from $75 to $120 million. Cattle with clinical or sub-clinical BRD do not gain weight or produce milk as well as healthy animals. Beef cattle with BRD gain less weight, have reduced feed efficiency and often produce a lower grade carcass at slaughter. Perino L. J., Apley M., Bovine Respiratory Disease, in CURRENT VETERINARY THERAPY 4 (FOOD ANIMAL PRACTICE), 4^{TH} ED. 446-455 (Howard J. L., Smith R. A., eds., 1999). A direct correlation between pulmonary lesions observed at slaughter and reduced weight gains has been established in cattle with sub-clinical infections. Whittem T. E. et al., J. Am. Vet. Med. Assoc., 209:814-818 (1996).

In addition to the production losses associated with mortality and morbidity, significant costs are associated with the treatment of BRD due to the costs of various therapeutic agents and the labor required to administer these agents, along with the extra labor to isolate and observe these animals.

The pathogenesis of BRD is thought to be due to the interaction of environmental and physiological stresses coupled with infectious agents. Mannheimia (Pasteurella) haemolytica, Pasteurella multocida and Haemophilus somnus are considered part of the normal flora of the bovine upper respiratory tract. When environmental and physiological stress factors reduce the natural resistance and inhibit the pulmonary defense mechanisms these organisms proliferate and colonize the lower respiratory tract. In addition, various bovine viruses such as infectious bovine rhinotracheitis virus (IBRV), bovine viral diarrhea virus (BVDV), bovine respiratory syncytial virus (BRSV) and parainfluenza 3 virus (PI-3) are known to have immunosuppressive effects in the lung.

Similarly, swine respiratory disease (SRD) also has a multifactional etiology. Bacterial infections caused by P. multocida, H. parasuis, Bordetella bronchiseptica, Actinobacillus pleuropneumoniae, Streptococcus suis, Salmonella cholerasuis and Mycoplasma sp. can result in respiratory disease in swine, resulting in significant economic losses. Stresses such as crowding, mixing and moving of pigs and transient viral infections can contribute to the intensification of the disease.

For years antimicrobial therapy has been the mainstay of BRD therapy. There are many effective antimicrobial agents currently available for the treatment of BRD. NUFLOR®, an injectable formulation of the broad spectrum antibiotic Florfenicol, has emerged as one of the leading antibiotics on a global basis. It is indicated for the treatment and control of BRD associated with M. haemolytica, P. multocida and H. somnus as well as for the prevention of respiratory disease in cattle at high risk of developing BRD associated with these bacteria. NUFLOR® is also indicated for the treatment of bovine interdigital phlegmon (footrot, acute interdigital necrobacillosis, infectious pododermatitis) associated with Fusobacterium necrophorum and Bacteroides melaninogenicus. NUFLOR® may be administered subcutaneously as well as intramuscularly.

The above mentioned products are typically administered as a single active component formulation—typically by some form of injection, pour on formulation or by oral administration. When the formulation is injectable, there are certain considerations that need to be taken into account. Multiple injections at the same or different sites can lead to local inflammation and irritation. Additionally, as the subject to be treated often must be caught and handled, there is an increase in the labor involved in administering the medication. Thus, it would be beneficial to use a formulation that contained both an antibiotic in combination with an antiparasitic agent to remedy the aforementioned problems. Accordingly, there is a need for conveniently administered, stable compositions that can control and prevent the infections associated with bovine respiratory disease and other infectious diseases as well as control and prevent parasitic infections.

### SUMMARY OF THE INVENTION

The present invention provides improved compositions and methods for the treatment of respiratory disease, parasitic infection, parasitic infestation, bacterial infection and other infections of cattle and other animals.

Accordingly, there is disclosed a composition for the treatment of microbial and parasitic infection in an animal comprising

a) a compound selected from the group consisting of

a compound of Formula I:

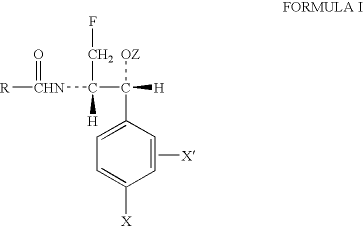

wherein R is a member selected from the group consisting of methyl or ethyl or a halogenated derivative thereof, dihalogenodeuteriomethyl, 1-halogeno-1-deuterioethyl, 1,2-dihalogeno-1-deuterioethyl, azidomethyl and methylsulfonylmethyl;

each of X and X′ is a member independently selected from the group consisting of NO₂, SO₂R₁, SOR₁, SR₁, SONH₂, SO₂NH₂, SONHR₁, SO₂NHR₁, COR₁, OR₁, R₁, CN, halogen, hydrogen, phenyl, and phenyl substituted by halogen, NO₂, R₁, PO₂R₁, CONHR₁, NHR₁, NR₁R₂, CONR₁R₂, OCOR₁, or OR₁, wherein each of R₁ and R₂ is a member independently selected from the group consisting of methyl, ethyl, n-propyl, isopropyl butyl, t-butyl, isobutyl and phenyl;

and Z is hydrogen or an acyl group of a hydrocarboncarboxylic acid having up to 16 carbon atoms or an acyl group of an aminohydrocarboncarboxylic acid having up to 12 carbon atoms; and the pharmaceutically acceptable salts of said acyl groups; b) an endectocidic compound possessing antiparasitic activity; and c) a carrier.

There is also disclosed a composition for the treatment of a microbial and parasitic infection in an animal comprising: a) a macrolide antibiotic selected from the group consisting of Tilmicosin and Tulathromycin; b) an endectocidic compound possessing antiparasitic activity; and c) a carrier.

There is also disclosed a composition for the treatment of a microbial and parasitic infection in an animal comprising: a) a cephalosporin selected from the group consisting of Ceftiofur and Cefquinome; b) an endectocidic compound possessing antiparasitic activity; and c) a carrier.

There is also disclosed a composition for the treatment of a microbial and parasitic infection in an animal comprising: a) a fluoroquinolone antibiotic selected from the group consisting of Enrofloxacin, Danofloxacin and Marbofloxacin; b) an endectocidic compound possessing antiparasitic activity; and c) a carrier.

There are also disclosed methods of using the formulations of the present invention to treat bacterial and parasitic based infections.

### DETAILED DESCRIPTION OF THE INVENTION

The invention provides novel compositions for the treatment of infectious diseases such as bovine respiratory disease in livestock as well as parasitic infections and infestations. These compositions are formulations comprising an antiparasitic compound, preferably an avermectin, in combination with certain antibacterial drugs, such as, for example, Florfenicol, Tilmicosin, Tulathromycin, Ceftiofur, Cefquinome, Enrofloxacin, Marbofloxacin or Danofloxacin.

The following terms will be defined as is known to one of skill in the art. “Acyl” means an H—C(O)—, alkyl-C(O)—, alkenyl-C(O)—, alkynyl-C(O)—, cycloalkyl-C(O)—, cycloalkenyl-C(O)—, or cycloalkynyl-C(O)— group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, butanoyl and cyclohexanoyl.

“Alkyl” means an aliphatic hydrocarbon group, which may be straight or branched, comprising from 1 to about 20 carbon atoms in the chain. Preferred alkyl groups contain from 1 to about 12 carbon atoms in the chain. More preferred alkyl groups contain from 1 to about 6 carbon atoms in the chain. Branched means that one or more lower alkyl groups, such as methyl, ethyl or propyl, are attached to a linear alkyl chain. “Lower alkyl” means a group having from 1 to about 6 carbon atoms in the chain, which may be straight or branched. The term “substituted alkyl” means that the alkyl group may be substituted by one or more substituents which may be the same or different.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents,” which may be the same or different, and are as defined herein.

“Alkoxy” means an alkyl-O— group in which the alkyl group is as previously described. Non-limiting examples of suitable alkoxy groups include methoxy, ethoxy, isopropoxy, and n-butoxy. The bond to the parent moiety is through the ether oxygen.

“Azido” refers to an —N₃ group.

“Halo” and “halogeno” mean fluoro, chloro, bromo, or iodo groups. Preferred are fluoro, chloro or bromo, and more preferred are fluoro and chloro.

“Halogen” means fluorine, chlorine, bromine, or iodine. Preferred are fluorine, chlorine or bromine, and more preferred are fluorine and chlorine.

“Haloalkyl” and “halogenoalkyl” mean an alkyl group as defined above wherein one or more hydrogen atoms on the alkyl is replaced by a halo group defined above.

“Ring system substituent” means a substituent attached to an aromatic or non-aromatic ring system which, for example, replaces an available hydrogen on the ring system. Ring system substituents may be the same or different.

The term “optionally substituted” means optional substitution with the specified groups, radicals or moieties.

As used herein, the term “composition” is intended to encompass a product comprising the specified ingredients in the specified amounts, as well as any product which results, directly or indirectly, from combination of the specified ingredients in the specified amounts.

An “effective amount” is the dose required to alleviate a particular symptom of an infection, infestation or disease or to protect an animal against infections, infestations or diseases.

As used herein, the term “bovine” refers to animals of the genus Bos, such as cattle. The term “bovid” refers to animals in the family Bovidae, which includes hoofed, hollow-horned ruminants such as cattle, sheep, goats, buffaloes, oxen, etc. As used herein, the term “swine” refers to animals of the family Suidae, which includes pigs, boars, warthogs, etc.

Fluorine-containing analogs of antibiotics chloramphenicol and thiamphenicol have been shown to have antibiotic activity, both against organisms sensitive to and resistant to chloramphenicol and thiamphenicol. See Schafer, T. W. et al., “Novel Fluorine-Containing Analogs of Chloramphenicol and Thiamphenicol: Antibacterial and Biological Properties,” in CURRENT CHEMOTHERAPY AND INFECTIOUS DISEASE PROCEEDINGS OF THE 11^{TH} ICC AND THE 19^{TH} ICMC AMERICAN SOClETY OF MICROBIOLOGY 1980, 444-446. Examples of such compounds, and methods for their manufacture, are described and claimed in U.S. Pat. No. 4,235,892. The medical profession has become increasingly concerned about the transference of bacterial resistance to humans when antibiotics useful in treating humans are administered to livestock. Because the chloramphenicol group of antibiotics is infrequently used now to treat humans, its derivatives are particularly appropriate for veterinary use. Of particular interest are the 3-fluoro, 3-deoxy derivatives.

The compositions of the present invention comprise an antiparasitic compound, preferably an avermectin, and at least one antibiotic of Formula I:

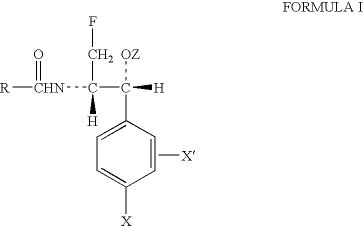

wherein R is a member selected from the group consisting of methyl or ethyl or a halogenated derivative thereof, dihalogenodeuteriomethyl, 1-halogeno-1-deuterioethyl, 1,2-dihalogeno-1-deuterioethyl, azidomethyl and methylsulfonylmethyl;

each of X and X′ is a member independently selected from the group consisting of NO₂, SO₂R₁, SOR₁, SR₁, SONH₂, SO₂NH₂, SONHR₁, SO₂NHR₁, COR₁, OR₁, R₁, CN, halogen, hydrogen, phenyl, and phenyl substituted by halogen, NO₂, R₁, OR₁, PO₂R₁, CONHR₁, NHR₁, NR₁R₂, CONR₁R₂ or OCOR₁, wherein each of R₁ and R₂ is a member independently selected from the group consisting of methyl, ethyl, n-propyl, isopropyl, butyl, t-butyl, isobutyl and phenyl;

and Z is hydrogen or an acyl group of a hydrocarboncarboxylic acid (preferably a hydrocarbondicarboxylic acid) having up to 16 carbon atoms or an acyl group of an amino-hydrocarboncarboxylic acid having up to 12 carbon atoms; and the pharmaceutically acceptable salts of said acyl groups.

Included among the halogenated groups contemplated for the moiety R in Formula I are the mono-, di- and tri-fluoro, the mono-, di- and tri-chloro-, the mono- and di-bromo-, and the iodo-methyl groups as well as the mono- and di-fluoro-, the mono- and di-chloro-, the mono- and di-bromo-, and the iodo-ethyl groups wherein the halogen substituents are preferably on the carbon alpha to the carbonyl function. Also included are mixed dihalogenoalkyl groups in which both halogens are preferably bonded to the carbon alpha to the carbonyl groups, e.g., groups such as fluorochloro-, fluorobromo-, and chlorobromo-methyl and -ethyl, as well as trihalogen-methyl groups such as dichlorofluoro- and difluorochloromethyl.

Also included among the compounds of Formula I are the ester derivatives, e.g. 1-hydrocarboncarboxylates of Formula I wherein Z is an acyl group of a hydrocarboncarboxylic acid having up to 16 carbon atoms that may be saturated, unsaturated, straight chain or branched chain, aliphatic, cyclic, cyclic-aliphatic, aromatic, aryl-aliphatic, or alkyl-aromatic and may be substituted by hydroxy, alkoxy containing from 1 to 5 carbon atoms, carboxyl, NO₂, NHR₁, NR₁R₂, SR₁, SOR₁, or halogen, wherein R₁ and R₂ are as defined above.

Other antibacterially active ester derivatives of Formula I are those wherein Z is an acyl group of an amino acid containing up to 12 carbon atoms that may be saturated, unsaturated, straight chain, branched chain or cyclic, that may contain aromatic groups and that may be substituted by hydroxyl groups.

Preferred ester derivatives include those derived from dibasic hydrocarboncarboxylates, e.g. the 1-succinate and 1-palmitate esters, which provide water soluble, pharmaceutically acceptable cationic salts, e.g. the sodium or potassium salts as well as salts with amine, e.g. trimethylamine. Also preferred are ester derivatives of amino acids that provide water soluble, pharmaceutically acceptable acid addition salts with mineral or organic acids, e.g. the hydrochloric, or sulfuric acid, or succinic acid addition salts.

As used herein the term “pharmaceutically acceptable salts” thus includes salts wherein the acidic hydrogen in the dibasic hydrocarboncarboxylate esters of this invention is replaced with a cation (e.g. sodium D-(threo)-1-p-nitrophenyl-2-dichloroacetamido-3-fluoro-1-propyl hemisuccinate) as well as salts wherein the acidic hydrogen forms an acid addition salt with an amine (e.g. D-(threo)-1-p-nitrophenyl-2-dichloroacetamido-3-fluoro-1-propyl hemisuccinate N-trimethylamine salt). Also included are the acid addition salts formed between mineral or organic acids and the amine in the amino acid esters of the compounds of Formula I (e.g.

D-(threo)-1-p-nitrophenyl-2-dichloroacetamido-3-fluoro-1-propyl glycinate hydrochloride).

Among the pharmaceutically acceptable cationic salts of the dibasic hydrocarboncarboxylate esters included in Formula I are salts of alkali and alkaline earth metals (e.g., sodium, potassium, calcium, aluminum) and salts with an amine such as trialkylamines, procaine, dibenzylamine, N-benzyl-beta-phenethylamine, N,N′-dibenzylethylenediamine, N-(lower)alkylpiperidines (e.g. N-ethylpiperidine), and N-methyl glucamine.

Preferably R is a halogenated derivative of methyl or ethyl, Z is a hydrogen, X is phenyl, COR₁ or SO₂R₁, R₁ is methyl, and X′is hydrogen. Most preferably R is CHCl₂ or CHF₂.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

A preferred antibiotic compound is Florfenicol, also known as (D-(threo)-1-p-methylsulfonyl phenyl-2-dichloroacetamido-3-fluoro-1-propanol). Another preferred antibiotic compound is D-(threo)-1-p-methylsulfonyl phenyl-2-difluoroacetamido-3-fluoro-1-propanol. Another preferred antibiotic is Thiamphenicol. Processes for the manufacture of these preferred antibiotic compounds, and intermediates useful in such processes, are described in U.S. Pat. Nos. 4,311,857; 4,582,918; 4,973,750; 4,876,352; 5,227,494; 4,743,700; 5,567,844; 5,105,009; 5,382,673; 5,352,832; and 5,663,361. When the antibiotic compound is Florfenicol, the concentration of Florfenicol typically is from about 10% to about 50% w/v, with the preferred level between about 20% and about 40% w/v, even more preferred being at least about 30% w/v.

Another preferred antibiotic compound is Tilmicosin. Tilmicosin is a macrolide antibiotic that is chemically defined as 20-dihydro-20-deoxy-20-(cis-3,5-dimethylpiperidin-1-yl)-desmycosin and which is reportedly disclosed in U.S. Pat. No. 4,820,695. Also disclosed in U.S. Pat. No. 4,820,695 is an injectable, aqueous formulation comprising 50% (by volume) propylene glycol, 4% (by volume) benzyl alcohol, and 50 to 500 mg/ml of active ingredient. Tilmicosin may be present as the base or as a phosphate. Tilmicosin has been found to be useful in treatment of respiratory infections, particularly Pasteurella haemolytica infections in cattle when administered by injection over a 4 day treatment period. Accordingly, Tilmicosin may be used in treatment of, for example, neonatal calf pneumonia and bovine respiratory disease. When Tilmicosin is present, it is present in an amount of about 1% to about 50%, preferably 10% to about 50%, preferably 30%.

Another suitable antibiotic for use in the present invention is Tulathromycin. Tulathromycin has the following chemical structure:

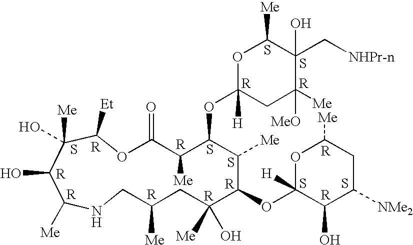

Tulathromycin may be identified as 1-Oxa-6-azacyclopentadecan-15-one, 13-[[2,6-dideoxy-3-C-methyl-3-O-methyl-4-C-[(propylamino)methyl]-α-L-ribo-hexopyranosyl]oxy]-2-ethyl-3,4, 10-trihydroxy-3,5,8,10,12,14-hexamethyl-11-[[3,4,6-trideoxy-3-(dimethylamino)-β-D-xylo-hexopyranosyl]oxy]-, (2R, 3S, 4R, 5R, 8R, 10R, 11 R, 12S, 13S, 14R). Tulathromycin may be prepared in accordance with the procedures set forth in U.S. Publication No. 2003/0064939 A1, which is incorporated by reference in its entirety. Tulathromycin may be present in injectable dosage forms at concentration levels ranging from about 5.0% to about 70% by weight. Tulathromycin is most desirably administered in dosages ranging from about 0.2 mg per kg body weight per day (mg/kg/day) to about 200 mg/kg/day in single or divided doses (i.e., from 1 to 4 doses per day), and more preferably 1.25, 2.5 or 5 mg/kg once or twice weekly, although variations will necessarily occur depending upon the species, weight and condition of the subject being treated. Tulathromycin may be present in injectable dosage forms at concentration levels ranging from about 5.0% to about 70% by weight.

Also preferred antibiotics for use in the present invention include cephalosporins such as, for example, Ceftiofur, Cefquinome, etc. The concentration of the cephalosporin in the formulation of the present invention may vary between about 1 mg/ml to 500 mg/ml.

Also preferred antibiotics include fluoroquinolones, such as, for example, Enrofloxacin, Danofloxacin, Difloxacin, Orbifloxacin and Marbofloxacin. In the case of Enrofloxacin, it may be administered in a concentration of about 100 mg/ml. Danofloxacin may be present in a concentration of about 180 mg/ml.

Other preferred macrolide antibiotics include compounds from the class of ketolides, or, more specifically, the azalides. Such compounds are described in, for example, U.S. Pat. Nos. 6,514,945, 6,472,371, 6,270, 768, 6,437,151 and 6,271,255, assigned to Pfizer, and U.S. Pat. Nos. 6,239,112, 5,958,888, assigned to Merial, and U.S. Pat. Nos. 6,339,063 and 6,054,434, assigned to Merck & Co., all of which are incorporated by reference in their entirety.

Other antibiotics may include tetracyclines, particularly Chlortetracycline and Oxytetracycline. Other antibiotics may include p-lactams such as penicillins, e.g., Penicillin, Ampicillin, Amoxicillin, or a combination of Amoxicillin with Clavulanic acid or other beta lactamase inhibitors

Additionally, the present invention may encompass a composition for the treatment of a microbial and parasitic infection in an animal comprising:a) oxytetracycline; b) an endectocidic compound possessing antiparasitic activity such as Ivermectin, Doramectin, Abamectin, Selamectin, Emamectin, Eprinomectin, Moxidectin and Milbemycin; and c) at least one carrier.

Antiparasitic compounds useful within the scope of the present invention are preferably comprised of the class of avermectin compounds. As stated above, the avermectin family of compounds is a series of very potent antiparasitic agents known to be useful against a broad spectrum of endoparasites and ectoparasites in mammals. The compositions of the present invention are useful against both internal parasitic infection and external parasitic infestation.

A preferred compound for use within the scope of the present invention is Ivermectin. Ivermectin is a semi-synthetic derivative of avermectin and is generally produced as a mixture of at least 80% 22,23-dihydroavermectin B1ₐ and less than 20% 22,23-dihydroavermectin B1_{b}. Ivermectin is disclosed in U.S. Pat. No. 4,199,569, hereby incorporated by reference. Ivermectin has been used as an antiparasitic agent to treat various animal parasites and parasitic diseases since the mid-1980's.

Abamectin is an avermectin that is disclosed in U.S. Pat. No. 4,310,519, the entirety of which is incorporated herein by reference, as avermectin B1a/B1 b. Abamectin contains at least 80% of avermectin B1ₐ and not more than 20% of avermectin B1_{b}.

Another preferred avermectin is Doramectin also known as 25-cyclohexyl-avermectin B₁. The structure and preparation of Doramectin, is disclosed in U.S. Pat. No. 5,089,480, which is herein incorporated by reference.

Another preferred avermectin is Moxidectin. Moxidectin, also known as LL-F28249 alpha is known from U.S. Pat. No. 4,916,154, which is herein incorporated by reference.

Another preferred avermectin is Selamectin. Selamectin is 25-cyclohexyl-25-de(1-methylpropyl)-5-deoxy-22,23-dihydro-5-(hydroxyimino) avermectin B₁ monosaccharide.

Milbemycin, or B41, is a substance which is isolated from the fermentation broth of a Milbemycin producing strain of Streptomyces. The microorganism, the fermentation conditions and the isolation procedures are more fully described in U.S. Pat. No. 3,950,360 and U.S. Pat. No. 3,984,564.

Emamectin (4″-deoxy4″ epimethylaminoavermectin B.sub.1), which can be prepared as described in U.S. Pat. No. 5,288,710 or 5,399,717, is a mixture of two homologues, 4″-deoxy4″-epi-methylaminoavermectin B1a and 4″-deoxy4″-epi-methylaminoavermectin B1b. Preferably, a salt of Emamectin is used. Non-limiting examples of salts of Emamectin which may be used in the present invention include the salts described in U.S. Pat. No. 5,288,710, e.g., salts derived from benzoic acid, substituted benzoic acid, benzenesulfonic acid, citric acid, phosphoric acid, tartaric acid, maleic acid, and the like. Most preferably, the Emamectin salt used in the present invention is Emamectin benzoate.

Eprinomectin is chemically known as 4″-epi-Acetylamino-4″-deoxy-avermectin B₁. Eprinomectin was specifically developed to be used in all cattle classes and age groups. It was the first avermectin to show broad-spectrum activity against both endo- and ecto-parasites while also leaving minimal residues in meat and milk. It has the additional advantage of being highly potent when delivered topically.

The compositions of the present invention may also further comprise a flukicide. Suitable flukicides include, for example, Triclabendazole, Fenbendazole, Albendazole, Clorsulon and Oxibendazole. It will be appreciated that the above combinations may further include combinations of antibiotic, antiparasitic and anti-fluke active compounds.

The formulations of the present invention may be administered by injection. In addition to greater convenience and ease of use with a combination product formulation, it is believed that a single daily subcutaneous administration of a combination product in accordance with the present invention will promote humane animal care by reducing the number of injections needed to treat animals. By reducing the number of injections, manpower costs also may be significantly reduced. Alternatively, the formulations of the present invention may be administered as a pour on solution. In another embodiment, the formulations of the present invention may be administered, for example, orally, such as a feed additive or a paste, parenterally, such as by intravenous administration.

The remaining portion of the formulations of the present invention is a pharmaceutically acceptable carrier comprising at least one solvent. The pharmaceutically acceptable carrier comprises from about 15% to about 80% of the formulation.

Florfenicol is generally soluble in aprotic polar solvents such as a pyrrolidone solvent, or N,N-dimethylacetamide, N,N-dimethylformamide, DMSO, acetone or glycerol formal. Preferred pyrrolidone solvents are N-methyl-2-pyrrolidone and 2-pyrrolidone. Accordingly, such an aprotic polar solvent (or a combination of such solvents) is preferred for use in formulations of the present invention that contain Florfenicol or similar antibiotics. Preferably such a solvent is present at about 5% to about 80% by weight of the formulation. More preferably such a solvent is present at about 10% to about 35% of the formulation.

Other pharmaceutically acceptable solvents may be present in the formulations of the present invention. Suitable solvents include water, ethanol, isopropanol, 1,2-propanediol, glycerin, benzyl alcohol, triacetin dimethylisosorbide, dimethylisosorbide, triacetin, glycol ethers, monothioglycerol, propylene glycol and polyethylene glycol (PEG). Particularly preferred solvents include PEG having an average molecular weight between about 200 and about 400, triacetin, dimethylisosorbide, ethanol, and water, and combinations thereof. These solvents may comprise from 0% to about 75% of the formulation. Preferably they comprise from about 15% to about 60%. More preferably they comprise from about 40% to about 55% of the formulation.

The addition of one or more of such additional solvents may be desirable to reduce the viscosity of the formulation in order to provide a product with workable syringeability. Examples of solvents particularly useful for adjusting the viscosity of the formulations of the present invention include water, ethanol, isopropanol, propylene glycol, dimethylisosorbide and triacetin, and combinations thereof.

Other inert ingredients can be added to the present composition, as desired. Such ingredients include preservatives, chelating agents, antioxidants and stabilizers. Exemplary preservatives include methyl p-hydroxybenzoate (methylparaben) and propyl p-hydroxybenzoate (propylparaben). Exemplary chelating agents include edetate sodium. Exemplary antioxidants include butylated hydroxyanisole and sodium monothioglycerol.

In order to prepare the composition of the present invention, the vehicle(s) or a portion of the vehicle(s), are added to the compounding vessel, followed by the remaining excipients and the actives. The mixture is mixed until all solids are dissolved. Additional solvent to bring the composition to final volume may be added if needed. Additives, such as those listed above, may also be included in the vessel and mixed into the formulation (the order of addition is not critical).

When the antibiotic is Florfenicol, the compositions according to the present invention will generally be administered to cattle at from about 1 mg to about 100 mg of the antibacterial per kilogram of body weight. Preferably the compositions of the present invention will be administered to bovines at from about 20 mg to about 50 mg of the antibacterial per kilogram of body weight. More preferably the dose will be about 40 mg/kg of the antibacterial. The compositions according to the present invention will generally be administered to swine at a dose of from 15 mg to about 100 mg of the antibacterial per kilogram of body weight. Preferably the compositions of the present invention will be administered to swine at from about 20 mg to about 50 mg of the antibacterial per kilogram of body weight.

Preferably, when the antibiotic is tilmicosin, the dose of Tilmicosin would be about 10 milligrams/kilogram. Tulathromycin is most desirably administered in dosages ranging from about 0.2 mg per kg body weight per day (mg/kg/day) to about 200 mg/kg/day in single or divided doses (i.e., from 1 to 4 doses per day), and more preferably 1.25, 2.5 or 5 mg/kg as a single dose, although variations will necessarily occur depending upon the species, weight and condition of the subject being treated.

Preferably, for Ceftiofur hydrochloride, the concentration is about 50 mg/ml. It may be administered as 1 to 2.2 mg/kg of body weight by intramuscular or subcutaneous injection, at 24-hour intervals for 3 to 5 consecutive days.

For single-dose therapy, Enrofloxacin may be administered 7.5 to 12.5 milligrams Enrofloxacin per kilogram of body weight. For multiple-day therapy it may be administered 2.5 to 5.0 milligrams per kilogram of body weight administered subcutaneously once daily for 3 to 5 days. In the case of Danofloxacin, it may be administered in a concentration of about 180 mg/ml at a dose of 6 mg/kg body weight in a single or multiple dose.

For cattle, administer 10 milligrams per 50 kilograms of the endectocides. It is used in cattle for the treatment and control of gastrointestinal nematodes (adults and fourth-stage larvae) (Haemonchus placei, Ostertagia ostertagi (including inhibited larvae) O. lyrata, Trichostrongylus axei, T. colubriformis, Cooperia oncophora, C. punctata, C. pectinata, Oesophagostomum radiatum, Nematodirus helvetianus (adults only), N. spathiger (adults only), Bunostomum phlebotomum); lungworms (adults and fourth-stage larvae) (Dictyocaulus viviparus); grubs (first, second, and third instars) (Hypoderma bovis, H. lineatum); lice (Linognathus vituli, Haematopinus eurystemus, Solenopotes capillatus); mites (Psoroptes ovis (syn. P. communis var. bovis), Sarcoptes scabiei var. bovis). It is also used to control infections of D. viparus for 28 days after treatment and O. ostertagi for 21 days after treatment, and H. placei, T. axei, C. punctata, C. oncophora, and Oesophagostomum radiatum for 14 days after treatment.

When Doramectin is present for use in cattle, administer 200 micrograms per kilogram (10 milligrams per 110 pounds) as a single subcutaneous or intramuscular injection. Doramectin is indicated for the treatment and control of gastrointestinal roundworms, lungworms, eyeworms, grubs, sucking lice, and mange mites. To control infections and to protect from reinfection with Cooperia oncophora and Haemonchus placei for 14 days, Ostertagia ostertagi for 21 days, and C. punctata, Oesophagostomum radiatum, and Dictyocaulus viviparus for 28 days after treatment. For swine, administer 300 micrograms per kilogram (10 milligrams per 75 pounds as a single intramuscular injection. It is indicated for treatment and control of gastrointestinal roundworms, lungworms, kidney worms, sucking lice, and mange mites.

Other doses for other antihelminthic compounds may be ascertained by one of ordinary skill in the art.

The compositions may be administered once daily or divided into multiple doses. Often only one dose will be sufficient to treat the infection. In some circumstances one dose followed by a second dose 48 hours later will be required to treat the animal. The precise dose will depend on the stage and severity of the infection, the susceptibility of the infecting organism to the composition, and the individual characteristics of the animal species being treated, as will be appreciated by one of ordinary skill in the art.

The compositions according to the present invention are particularly useful for cattle and other bovids, swine, and other large mammals. In addition to the treatment of bovine respiratory disease, the compositions of this invention are also suitable for the treatment of infectious such as swine respiratory disease, footrot, acute mastitis, pinkeye (infectious keratoconjunctivitis), acute pneumonia, metritis and enteritis. The dosage regimen for treatment of such diseases would be as described above.

Pinkeye is an acute infectious disease of cattle, sheep and other animals that is characterized by inflammation of the tissues of the eye, accompanied by nasal discharge, lacrimation and copious ocular discharge. Affected animals may display extreme discomfort, resulting in a drop in milk production; in extreme cases permanent blindness occurs. The disease, which is caused by Moraxella bovis in cattle, is widespread, especially among range and feedlot cattle, and is of great economic importance to the cattle industry.

Footrot (interdigital phlegmon) is an acute infection of the interdigital space that occurs throughout the world in both beef and dairy cattle. Fusobacterium necrophorum is the major cause of footrot, although other organisms, including Bacteroides melaninogenicus, can be involved. The major symptoms include pain, severe lameness, fever, anorexia, and reduced milk production.

Currently, footrot is treated by antibiotic therapy; recommended therapy can involve treatment for up to five days. The use of the formulations of the present invention for the treatment of footrot would be an improvement over presently known treatments because it would provide the proven efficacy of Florfenicol (with fewer administrations). The compositions of the present invention are also useful for the prevention of these diseases in animals at high risk of developing those diseases. For example, the presently-claimed compositions can be administered to cattle at high risk of developing bovine respiratory disease at the same dosages recommended for treatment of bovine respiratory disease.

The invention will be set forth with more particularity by the following non-limiting examples.

### EXAMPLE 1

A formulation within the scope of the present invention was prepared according to procedures customary in the art. The formulation contained the following concentrations of ingredients as set forth in the table below.

| | Concentration |
|---|---|
| Ingredient | (mg/ml) |
| Florfenicol | 300 |
| Ivermectin | 1.5 |
| 2-Pyrollidone | 300 |
| BHT | 1 |
| Triacetin | qs |

The formulation demonstrated an acceptable stability profile when subjected to temperature cycling and assayed by high performance liquid chromatography as set forth in the table below.

| | | | % Ivermectin |
|---|---|---|---|
| | | % Florfenicol | Loss |
| | | Loss | (relative to the |
| Temperature | | (relative to RT | frozen sample at - |
| Condition | Duration | sample) | 10° C.) |
| 50° C. | 2.25 | 1.48 | — |
| | months | | |
| 70° C. | 9 days | — | 0.74 |

### EXAMPLE 2

The formulation prepared in accordance with Example 1 was administered to animals. The following study was conducted using the formulation prepared in accordance with Example 1. 10 calves were administered either the formulation of Example 1 administered as a single subcutaneous injection of Florfenicol dose of 40 mg/kg and an Ivermectin dose of 0.2 mg/kg or the calves were administered a single Ivermectin dose of 0.2 mg/kg. Mean serum concentrations were determined. The formulation of Example 1 was also compared against data previously obtained with the administration of commercially available formulations of Florfenicol as a single active agent. The formulations of Example 1 achieved acceptable mean serum levels obtained postdose as those obtained with the administration of commercially available formulations containing either Florfenicol or Ivermectin as single active agents.

Although certain presently preferred embodiments of the invention have been described herein, it will be apparent to those skilled in the art to which the invention pertains that variations and modifications of the described embodiments may be made without departing from the spirit and scope of the invention. Accordingly, it is intended that the invention be limited only to the extent required by the appended claims and the applicable rules of law.

We claim:

1. A composition for the treatment of microbial and parasitic infection in an animal comprising
a) a compound selected from the group consisting of
a compound of Formula I: 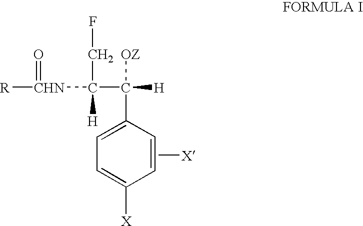
wherein R is a member selected from the group consisting of methyl or ethyl or a halogenated derivative thereof, dihalogenodeuteriomethyl, 1-halogeno-1-deuterioethyl, 1,2-dihalogeno-1-deuterioethyl, azidomethyl and methylsulfonylmethyl;
each of X and X′ is a member independently selected from the group consisting of NO₂, SO₂R₁, SOR₁, SR₁, SONH₂, SO₂NH₂, SONHR₁, SO₂NHR₁, COR₁, OR₁, R₁, CN, halogen, hydrogen, phenyl, and phenyl substituted by halogen, NO₂, R₁, PO₂R₁, CONHR₁, NHR₁, NR₁R₂, CONR₁R₂, OCOR₁, or OR₁, wherein each of R₁ and R₂ is a member independently selected from the group consisting of methyl, ethyl, n-propyl, isopropyl butyl, t-butyl, isobutyl and phenyl;
and Z is hydrogen or an acyl group of a hydrocarboncarboxylic acid having up to 16 carbon atoms or an acyl group of an aminohydrocarboncarboxylic acid having up to 12 carbon atoms; and the pharmaceutically acceptable salts of said acyl groups;
b) an endectocidic compound possessing antiparasitic activity; and
c) at least one carrier.

2. The composition of claim 1, wherein R is a halogenated derivative of methyl or ethyl.

3. The composition of claim 2, wherein R is CHCl₂ or CHF₂.

4. The composition of claim 3, wherein Z is hydrogen, X is SO₂R₁ or phenyl, and X′ is hydrogen.

5. The composition of claim 4, wherein R₁ is methyl.

6. The composition of claim 1, wherein the compound of Formula I is Florfenicol and the Florfenicol is present in an amount of about 10% w/v to about 50% w/v.

7. The composition of claim 1, wherein the endectocidic compound is an avermectin, and wherein the avermectin is a compound selected from the group consisting of Ivermectin, Doramectin, Abamectin, Selamectin, Emamectin, Eprinomectin, Moxidectin and Milbemycin.

8. The composition of claim 7, wherein the avermectin compound is present in an amount of about 0.03% w/v to about 20% w/v.

9. The composition of claim 7, wherein the avermectin compound is Ivermectin.

10. The composition of claim 1, wherein the at least one carrier is a solvent.

11. The composition of claim 10, wherein the solvent is present in an amount of about 15% w/v to about 80% w/v.

12. The composition of claim 10, wherein the solvent is selected from the group consisting of a pyrrolidone solvent, N,N-dimethylacetamide, N,N-dimethylformamide, DMSO, acetone, glycerol formal and combinations thereof.

13. The composition of claim 12, wherein the solvent is a pyrrolidone solvent selected from the group consisting of N-methyl-2-pyrrolidone, 2-pyrrolidone and combinations thereof.

14. The composition of claim 10 further comprising a second solvent.

15. The composition of claim 14, wherein the second solvent is selected from the group consisting of water, propylene glycol, polyethylene glycol, triacetin, dimethyl-isosorbide, ethanol, isopropanol, glycerin, 1,2-propanediol, glycol ethers, benzyl alcohol and combinations thereof.

17. A composition for the treatment of a microbial and parasitic infection in an animal comprising:
a) a macrolide antibiotic selected from the group consisting of Tilmicosin and Tulathromycin;
b) an endectocidic compound possessing antiparasitic activity; and
c) at least one carrier.

18. The composition of claim 17, wherein the endectocidic compound is an avermectin, and wherein the avermectin is a compound selected from the group consisting of Ivermectin, Doramectin, Abamectin, Selamectin, Emamectin, Eprinomectin, Moxidectin and Milbemycin.

19. The composition of claim 18, wherein the avermectin compound is present in an amount of about 0.03% w/v to about 20% w/v.

20. The composition of claim 17, wherein the at least one carrier is a solvent.

21. The composition of claim 20, wherein the solvent is present in an amount of about 15% w/v to about 80% w/v.

22. The composition of claim 21, wherein the solvent is selected from the group consisting of a pyrrolidone solvent, N,N-dimethylacetamide, N,N-dimethylformamide, DMSO, acetone, glycerol formal water, propylene glycol, polyethylene glycol, triacetin, dimethyl-isosorbide, ethanol, isopropanol, glycerin, 1,2-propanediol, glycol ethers, monothioglycerol, benzyl alcohol and combinations thereof.

23. The composition of claim 17 further comprising a fluckicide.

24. A composition for the treatment of a microbial and parasitic infection in an animal comprising:
a) a cephalosporin selected from the group consisting of Ceftiofur or Cefquinome;
b) an endectocidic compound possessing antiparasitic activity; and
c) at least one carrier.

25. The composition of claim 24, wherein the endectocidic compound is an avermectin, and wherein the avermectin is a compound selected from the group consisting of Ivermectin, Doramectin, Abamectin, Selamectin, Emamectin, Eprinomectin, Moxidectin and Milbemycin.

26. The composition of claim 25, wherein the avermectin compound is present in an amount of about 0.03% w/v to about 20% w/v.

27. The composition of claim 24, wherein the at least one carrier is a solvent, and wherein the solvent is selected from the group consisting of a pyrrolidone solvent, N,N-dimethylacetamide, N,N-dimethylformamide, DMSO, acetone, glycerol formal water, propylene glycol, polyethylene glycol, triacetin, dimethyl-isosorbide, ethanol, isopropanol, glycerin, 1,2-propanediol, glycol ethers, monothioglycerol, benzyl alcohol and combinations thereof.

28. The composition of claim 27, wherein the solvent is present in an amount of about 15% w/v to about 80% w/v.

29. The composition of claim 24 further comprising a fluckicide.

30. A composition for the treatment of a microbial and parasitic infection in an animal comprising:
a) a fluoroquinolone antibiotic selected from the group consisting of Enrofloxacin, Danofloxacin and Marbofloxacin;
b) an endectocidic compound possessing antiparasitic activity; and
c) at least one carrier.

31. The composition of claim 30, wherein the endectocidic compound is an avermectin, and wherein the avermectin is a compound selected from the group consisting of Ivermectin, Doramectin, Abamectin, Selamectin, Emamectin, Eprinomectin, Moxidectin and Milbemycin.

32. The composition of claim 31, wherein the avermectin compound is present in an amount of about 0.03% w/v to about 20% w/v.

33. The composition of claim 30, wherein the at least one carrier is a solvent, and wherein the solvent is selected from the group consisting of a pyrrolidone solvent, N,N-dimethylacetamide, N,N-dimethylformamide, DMSO, acetone, glycerol formal water, propylene glycol, polyethylene glycol, triacetin, dimethyl-isosorbide, ethanol, isopropanol, glycerin, 1,2-propanediol, glycol ethers, monothioglycerol, benzyl alcohol and combinations thereof.

34. The composition of claim 33, wherein the solvent is present in an amount of about 15% w/v to about 80% w/v.

35. The composition of claim 30 further comprising a fluckicide.

36. A method of treating bovine respiratory disease and a parasitic infection in an animal comprising the step of subcutaneously administering to an animal in need of such treatment a therapeutically effective amount of the composition of claim 1.

37. A method of treating bovine respiratory disease and a parasitic infection in an animal comprising the step of subcutaneously administering to an animal in need of such treatment a therapeutically effective amount of the composition of claim 17.

38. A method of treating bovine respiratory disease and a parasitic infection in an animal comprising the step of subcutaneously administering to an animal in need of such treatment a therapeutically effective amount of the composition of claim 24.

39. A method of treating bovine respiratory disease and a parasitic infection in an animal comprising the step of subcutaneously administering to an animal in need of such treatment a therapeutically effective amount of the composition of claim 30.
